(19) United States
(12) Patent Application Publication
(10) Pub. No.: US 2016/0120528 A1
(43) Pub. Date: May 5, 2016

(54) Hydrogel Pressure Sealant System

(71) Applicant: THE REGENTS OF THE UNIVERSITY OF CALIFORNIA, Oakland, CA (US)

(72) Inventor: Fereidoun Abtin, Los Angeles, CA (US)

(21) Appl. No.: 14/896,108

(22) PCT Filed: Jun. 5, 2014
(86) PCT No.: PCT/US14/41075

§ 371 Date: Dec. 4, 2015

Related U.S. Application Data
(60) Provisional application No. 61/831,375, filed on Jun. 5, 2013.

(51) Int. Cl. A61B 17/00; A61M 5/24; A61M 5/28; A61L 24/00; A61L 24/08

(52) U.S. Cl. CPC A61B 17/00491; A61L 24/0031; A61L 24/0015; A61L 24/08; A61M 5/284; A61M 5/2448; A61B 17/0057; A61L 2300/402; A61B 2017/00495; A61B 2017/0065; A61B 2017/00654; A61B 2017/00893

(57) ABSTRACT

The present invention includes devices, systems, and methods for sealing a defect in body tissue. For example, the present invention includes a device for preventing leakage of air and other gases from the lung during and after lung biopsy. The device delivers a hydrogel composition which forms an air-tight sealant. In certain embodiments, the device simultaneously delivers one or more therapeutic agents, such as lidocaine, to the treatment area.

### CROSS-REFERENCE TO RELATED APPLICATIONS

This application is a U.S. national phase application filed under 35 U.S.C. §371 claiming benefit to International Patent Application. No. PCT/US2014/041075, filed Jun. 5, 2014, which in turn is entitled to priority under 35 U.S.C. §119(e) to U.S. Provisional Application Ser. No. 61/831,375 filed on Jun. 5, 2013, the contents of which are incorporated by reference herein in its entirety.

### BACKGROUND OF THE INVENTION

Percutaneous transthoracic needle lung biopsy (a biopsy performed through the chest wall) has a risk of causing a pneumothorax, or collapsed lung, in a patient. Various studies have indicated that the rate of pneumothorax after fine needle aspiration of the lung was between 19% and 60% (Poe et al., 1984, Chest, 85: 232-235; Johnsrude et al., 1985, AJR Am J Roentgenol, 144: 793-794; Khouri et al., 1985, AJR Am J Roentgenol, 144: 281-288; van Sonnenberg et al., 1988, Radiology, 167: 457-461; Miller et al., 1988, Chest, 93: 742-745; Fish et al., 1988, AJR Am J Roentgenol, 150: 71-74; Hill et al., 1993, Chest, 104:1017-1020; Saji et al., 2002, Chest, 121: 1521-1526).

A collapsed lung occurs when air from the lung leaks into the space around it, preventing the lungs from expanding properly. The condition can cause chest pain, shortness of breath, and cardiovascular distress and is particularly dangerous in patients who have lung disease.

In clinical settings, prevention of pneumothorax is typically done by carefully performing the biopsy procedure, taking particular care of factors such as the angle of insertion, the number of needle passes, the dwell time of the needle in the lung, and the like. However, due to the variability in patients and in the health care providers performing the procedure, the risk of pneumothorax is continually present in such biopsy or aspiration procedures.

Therefore, there is a need in the art for a device and method to prevent pneumothorax during biopsy and other procedures. The present invention satisfies this unmet need.

### SUMMARY OF THE INVENTION

The present invention provides a device for sealing a tissue defect in a body tissue of a subject. The device comprises a first barrel and a first plunger engaged with the proximal end of the first barrel, thereby forming a first chamber within the first barrel, wherein the first chamber houses a first component of a hydrogel; a second barrel and a second plunger engaged with the proximal end of the second barrel, thereby forming a second chamber within the second barrel chamber, wherein the second chamber houses a second component of a hydrogel; and at least one port connecting the first chamber and second chamber. The contents of the first chamber and second chamber are temporarily isolated to prevent contact of the first component and second component prior to mixing.

In one embodiment, the port is configured for control by a user to allow communication between the first chamber and second chamber, thereby allowing contact of the first component and second component to form a hydrogel solution.

In one embodiment, the device comprises at least one outlet positioned on at least one of the first chamber and second chamber. In one embodiment, the outlet is configured to mate with a delivery instrument, such as a needle or a catheter.

In one embodiment, at least one of the first component and second component comprises one or more therapeutic agents. In one embodiment, the one or more therapeutic agents comprise one or more anesthetics.

In one embodiment, at least one of the first component and second component comprises a powderized and lyophilized hydrogel. In one embodiment, at least one of the first component and second component, comprises one or more of sodium alginate, hyaluronic acid, gelatin, fibrin, collagen, laminin, synthetic amphiphilic diblock copolypeptide, or agarose.

In one embodiment, the hydrogel solution forms a hydrogel capable of being injected to a target site of the subject. In one embodiment, the hydrogel solution forms a hydrogel, which remains at a target site of the subject, thereby sealing a tissue defect present at the target site.

The present provides a method of sealing a tissue defect at a target site of body tissue of a subject. The method comprises providing a device comprising a first barrel and a first plunger engaged with the proximal end of the first barrel, thereby forming a first chamber within the first barrel, wherein the first chamber houses a first component of a hydrogel; a second barrel and a second plunger engaged with the proximal end of the second barrel, thereby forming a second chamber within the second barrel chamber, wherein the second chamber houses a second component of a hydrogel; and at least one port connecting the first chamber and second chamber. The contents of the first chamber and second chamber are temporarily isolated to prevent contact of the first component and second component prior to mixing. The method further comprises mixing the first component and second component to form a hydrogel, attaching the device to a delivery instrument, guiding the delivery instrument to target site, and administering the hydrogel from the device, through the delivery instrument, onto the target site.

In one embodiment, the body tissue is selected from the group consisting of lung, liver, kidney, bone, and vasculature. In one embodiment, the tissue defect is a puncture wound formed during a clinical procedure.

In one embodiment, the administering of the hydrogel onto the target site is done prior to the development of a tissue defect at the target site.

In one embodiment, at least one of the first component and second component comprises one or more therapeutic agents. In one embodiment, the one or more therapeutic agents comprise one or more anesthetics.

In one embodiment, at least one of the first component and second component comprises a powderized and lyophilized hydrogel. In one embodiment, at least one of the first component and second component, comprises one or more of of sodium alginate, hyaluronic acid, gelatin, fibrin, collagen, laminin, synthetic amphiphilic diblock copolypeptide, and agarose.

In one embodiment, the administered hydrogel remains at a target site of the subject, thereby sealing a tissue defect present at the target site.

The present invention also provides a composition for sealing a tissue defect in a body tissue of a subject, where the composition comprising a hydrogel comprising 7% (wt/vol) sodium alginate and 1% (wt/vol) lidocaine.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1C, depicts an exemplary device of the invention prior to formulation of the hydrogel (FIG. 1A), during mixing (FIG. 1B), and following mixing prior to injection (FIG. 1C). The device depicted in FIG. 1 allows for the mixing of a liquid component in a first chamber with a solid component in a second chamber by opening of a distal port connecting the two chambers.

FIG. 2, comprising FIG. 2A through FIG. 2C, depicts another exemplary device of the invention prior to formulation of the hydrogel (FIG. 2A), during mixing (FIG. 2B), and following mixing prior to injection (FIG. 2C). The device depicted in FIG. 2 comprises a port positioned slightly proximally from the distal end, to allow for blockage of the port by a stopper, thereby isolating the two chambers.

FIG. 3, comprising FIG. 3A through FIG. 3C, depicts a set of side views (top) and perspective views (bottom) of three-dimensional representations of an exemplary device of the invention. The images depict the device prior to formulation of the hydrogel (FIG. 3A), during mixing (FIG. 3B), and following mixing prior to injection (FIG. 3C).

FIG. 4, comprising FIG. 4A and FIG. 4B, depicts an exemplary device of the invention prior to formulation of the hydrogel (FIG. 4A) and after mixing (FIG. 4B). The device depicted in FIG. 4 comprises a third chamber separating the first chamber, initially holding the solid component, and the second chamber, initially holding the liquid component.

FIG. 5, comprising FIG. 5A through FIG. 5C, depicts an exemplary device of the invention prior to formulation of the hydrogel (FIG. 5A), during mixing (FIG. 5B), and following mixing prior to injection (FIG. 5C). The device depicted in FIG. 5 comprises a third chamber, separating the first and second chambers, where the third chamber initially holds the solid component of the hydrogel.

FIG. 6, comprising FIG. 6A through FIG. 6C, is a set of CT images depicting an exemplary procedure of delivering a composition to the lung surface prior to needle biopsy of the lung. First, the needle is advanced to the pleural surface (FIG. 6A). The hydrogel is injected to provide pleural anesthesia and pressure seal (FIG. 6B). The needle and biopsy gun are advanced through the hydrogel to the tissue lesion (FIG. 6C).

FIG. 7 is an image depicting three compositions of the hydrogel comprising lidocaine.

FIG. 8 is a set of images depicting 3%, 4%, and 5% hyaluronic acid (HA) hydrogels.

FIG. 9 depicts an image of the hydrogel injected between the muscle and skin of chicken.

FIG. 10 is a graph depicting the stiffness (Pa) of various hydrogel formulations, as compared to native lung tissue and lidocaine solution alone.

FIG. 11, comprising FIG. 11A and FIG. 11B, depicts the experimental setup (FIG. 11A) and results (FIG. 11B) from experiments measuring the release profile of lidocaine from 1% lidocaine and 1% lidocaine in 10% gelatin.

FIG. 12, comprising FIG. 12A through FIG. 12C, depicts a set of CT images depicting the delivery of the hydrogel to the lung surface (FIG. 12A and FIG. 12B), as well as presence of the administered hydrogel 10 minutes post-delivery (FIG. 12C).

### DETAILED DESCRIPTION

The present invention includes devices, systems, and methods for sealing a defect in body tissue. For example, the present invention includes a device for preventing leakage of air and other gases from the lung during and after lung biopsy. The device delivers a hydrogel composition which forms an air-tight sealant. In certain embodiments, the device simultaneously delivers one or more therapeutic agents, such as lidocaine, to the treatment area.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to make, use, or perform the disclosed methods.

The term “biocompatible,” as used herein, refers to compositions that do not cause substantial toxicity, immune response or irritation to the surrounding tissue, to the extent that would prohibit a medical professional from using the composition on a patient.

The term “bioabsorbable,” as used herein refers to compositions which, once formed, slowly, e.g., during a period of days, weeks, or months, degrade and dissolve under normal physiological conditions.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides a device, system, and method for sealing of a tissue defect in a body tissue of a subject. For example, in certain embodiments, the present invention includes a hydrogel sealant, and devices, systems, and methods for application of a hydrogel sealant, for the treatment or prevention of wounds, punctures, cuts, burns, ulcers, and the like. In one embodiment, the invention provides for the treatment or prevention of a tissue defect in the lung, thereby preventing pneumothorax (i.e., collapsed lung) in a subject.

The present invention includes a device for applying a hydrogel sealant to a target site. In certain embodiments, the device of the invention delivers a hydrogel composition which forms an air-tight sealant at a target site within a subject. In certain embodiments, the device comprises a dual barrel syringe comprising a first chamber and a second chamber, where a first chamber holds a liquid component and a second chamber holds a solid component. In one embodiment, the device comprises a port connecting the first chamber and second chamber, such that immediately prior to use of the device, the liquid component and solid components are mixed together in the first chamber, second chamber, or both, to form a hydrogel solution. The hydrogel solution then gels to form a hydrogel prior to, during, or after using the device to administer the solution or gel to a target site within a subject.

In one embodiment, the invention includes a device and method for sealing of a lung puncture caused by biopsy, ablation, or other clinical procedures. In certain aspects, the hydrogel of the invention is applied to the surface of the lung prior to puncture of the lung during biopsy. The described invention reduces the risk of a patient experiencing a collapsed lung during a percutaneous transthoracic needle lung biopsy.

In one embodiment, the device and method of the invention are used to administer a varying stiffness pressure hydrogel to a body tissue before, during, or after a tissue defect is formed on or in the tissue. For example, in one embodiment, the device of the invention includes a hydrogel sealant for use in conjunction with lung needle biopsies, wherein the hydrogel is prepared and administered onto the surface of the lung before piercing the lung. The administered hydrogel thereby prevents air leakage into the pleural cavity and associated complications. In one embodiment, the device and method of the invention are used to administer a varying stiffness pressure hydrogel to a body tissue after a tissue defect is formed on or in the tissue to stop prolonged leak of air, blood, or other fluid.

In certain embodiments, the administered hydrogel of the invention releases a therapeutic agent, such as lidocaine, to the target site. Any therapeutic agent can be delivered, including, but not limited to, pain relievers, anesthetics, analgesics, steroids, hormones, anti-inflammatories, coagulants, anti-coagulants, and the like. In one embodiment, device of the invention is used to administer a hydrogel to the target site of the subject prior to a medical procedure, and can be used to release drugs such as pain relievers while providing a pressure seal against air or other fluids. The release of the one or more therapeutic agents may occur over seconds, minutes, hours, days, weeks, months, or years, from the initial application of the hydrogel.

In one embodiment, the device of the invention comprises a syringe assembly having at least two chambers. For example, the device may comprise a first chamber and a second chamber, where the first chamber houses a first component of a hydrogel and the second chamber holds a second component of a hydrogel, where prior to use of the device, the chambers are isolated thereby preventing contact of the first and second component. For example, in certain embodiment, the first and second chamber each houses one of a liquid component or a solid component of a hydrogel. These components are stored separately to provide an extended shelf life of the device, and to allow for a mixing mechanism for rapid hydration of the solid components into a hydrogel with various adjustable properties prior to application. Prior to application of a hydrogel to the tissue of the subject, the device is used to first formulate a hydrogel solution, by mixing the liquid and solid components together, and then to deliver the solution or polymerized hydrogel to a target site of the subject.

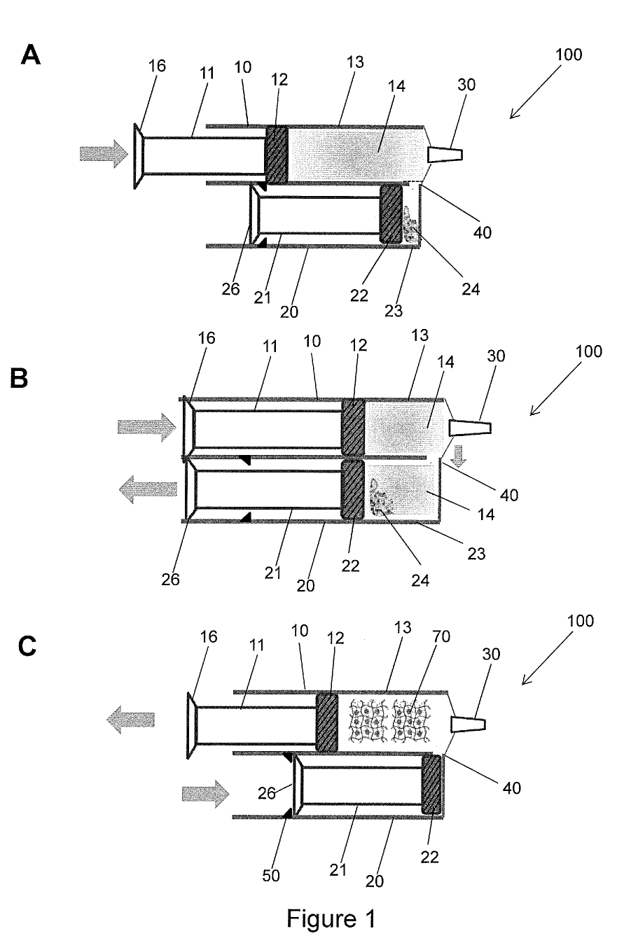

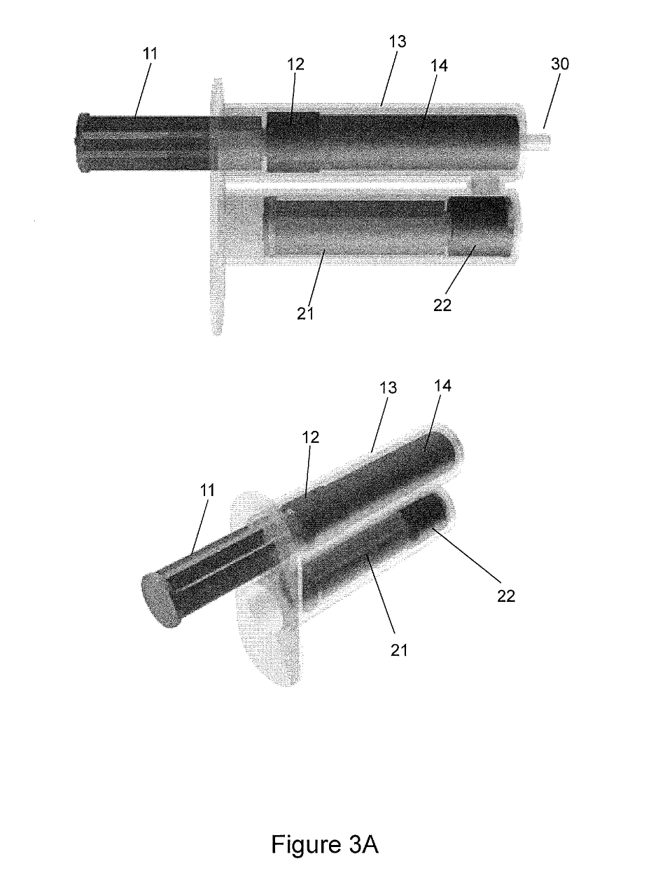

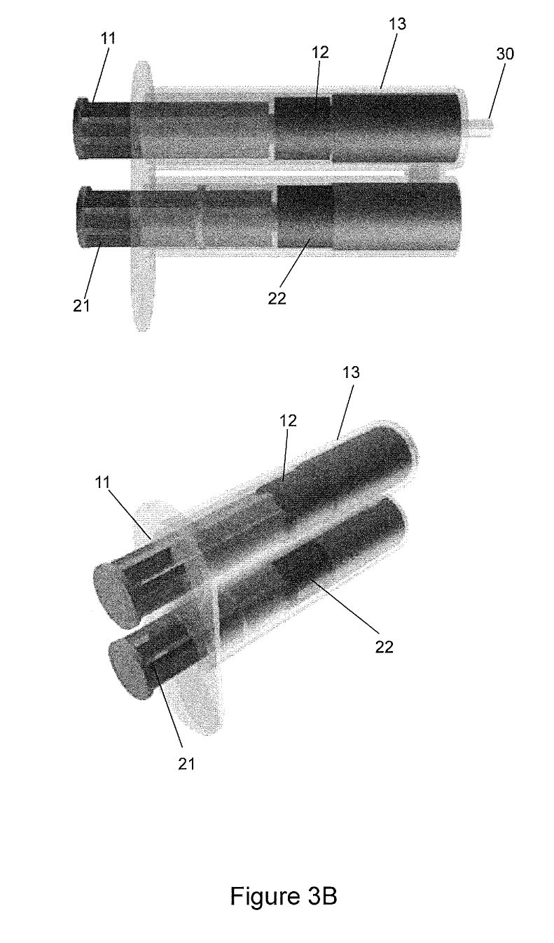

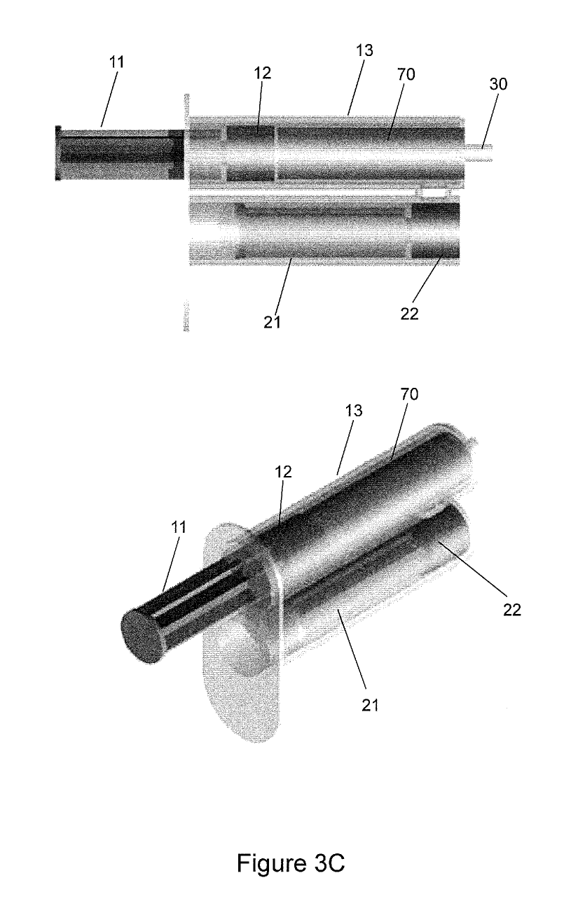

FIG. 1—FIG. 3 depict an exemplary device 100 of the invention, comprising a first barrel 10 and a second barrel 20. First barrel 10 comprises a proximal end, a distal end, and an inner bore or lumen running the length of the barrel. Device 100 comprises a first plunger 11 in communication with the proximal end of first barrel 10. For example, first plunger 11 is configured to be slidably engaged within first barrel 10, such that first plunger 11 may be manipulated to slide back and forth within the inner bore of first barrel 10. In certain embodiments, first plunger 11 comprises a handle 16 at the proximal end of first plunger 11 that allows push-pull motion of plunger 11 by a user. First plunger 11 further comprises first stopper 12 mounted to the distal tip of first plunger 11. First stopper 12 is designed to fit within first barrel 10 such that first stopper 12 fills the radial cross-section of first barrel 10, thereby forming a slidable seal within first barrel 10. First barrel 10 and first stopper 12 form a first chamber 13 positioned at the distal end of first barrel 10, such that sliding of first plunger 11 and first stopper 12 within first barrel 10 alters the size and volume of first chamber 13. First chamber 13 is capable of housing one or more components of the hydrogel, the hydrogel solution, and/or polymerized hydrogel.

Similar to first barrel 10, second barrel 20 comprises a proximal end, a distal end, and an inner bore or lumen running the length of the barrel. Device 100 comprises a second plunger 21 in communication with the proximal end of second barrel 20. For example, second plunger 21 is configured to be slidably engaged within second barrel 20, such that second plunger 21 may be manipulated to slide back and forth within the inner bore of second barrel 20. In certain embodiments, second plunger 21 comprises a handle 26 at the proximal end of second plunger 21 that allows push-pull motion of plunger 21 by a user. Second plunger 21 further comprises second stopper 22 mounted to the distal tip of second plunger 21. Second stopper 22 is designed to fit within second barrel 20 such that second stopper 22 fills the radial cross-section of second barrel 20, thereby forming a slidable seal within second barrel 20. Second barrel 20 and second stopper 22 form a second chamber 23 positioned at the distal end of second barrel 20, such that sliding of second plunger 21 and second stopper 22 within second barrel 20 alters the size and volume of second chamber 23. Second chamber 23 is capable of housing one or more components of the hydrogel, the hydrogel solution, and/or polymerized hydrogel.

In one embodiment, first chamber 13 comprises a first component of a hydrogel and second chamber comprises a second component of a hydrogel. For example, in one embodiment, first chamber 13 comprises liquid component 14 of the hydrogel, and second chamber 23 comprises a solid component 24 of the hydrogel. However, as would be understood by a skilled artisan, the device is not limited as to which chamber holds which component. For example, in certain embodiments, first chamber 13 holds the solid component, while second chamber 23 holds the liquid component. Prior to use of the device, first chamber 13 and second chamber 23 are used to hold and separate the liquid and solid components of the hydrogel, such that they come into contact only just prior to, or during, use of the device.

First barrel 10 and second barrel 20 may be of any suitable size and shape configured to hold an appropriate amount of liquid component 14 and solid component 24 within the chambers of each barrel. For example, the barrels may be cylindrical, rectangular, triangular, irregular shaped or the like.

In certain embodiments, first barrel 10 or second barrel 20 have a size suitable for housing a volume of liquid component 14 ranging from about 1 cc to about 100 cc, within first chamber 13 or second chamber 23. However, the device is not limited to any particular volume of liquid component 14. In certain embodiments, first barrel 10 or second barrel 20 have a size suitable for housing an amount of solid component 24 ranging from about 1 mg to about 100 g, within first chamber 13 or second chamber. However, the device is not limited to any particular amount or volume of solid component 24. The specified ranges allow for a wide tunable range of hydrogel parameters suitable for different physiologies and situations.

First barrel 10 and second barrel 20 may be manufactured from any suitable material known in the art, including but not limited to, plastic, polymer, glass, and the like. In certain embodiments, the material is non-reactive, such that it does not react with the liquid component, solid component, or hydrogel solution held within the chambers.

First stopper 12 and second stopper 22 may be manufactured from any suitable material known in the art, including but not limited to plastic, rubber, polymer, and the like. In certain embodiments, the material is non-reactive, such that it does not react with the liquid component, solid component, or hydrogel solution held within the chambers.

Device 100 further comprises a port 40 in communication with both first chamber 13 and second chamber 23. Port 40 may be of any suitable size and shape that allows passage of hydrogel components to and from first chamber 13 and second chamber 23. In certain aspects, port 40 may be an opening in a wall which separates first chamber 13 and second chamber 23. In another embodiment, port 40 comprises a conduit having a defined length, which connects first chamber 13 and second chamber 23. Port 40 is capable of providing controlled flow of liquid between first chamber 13 and second chamber 23 for the purposes of mixing solid component 24 and liquid component 14 to form a homogeneous hydrogel solution 70. Port 40 can control the flow of liquid in a variety of ways including but not limited to providing one-way, two-way, or manually adjustable flow control, using one or a combination of valves, membrane-based filters, shape-based mixing channels, or other flow control devices. In certain embodiments, port 40 comprises a removable physical barrier (dashed line), which separates the contents of first chamber 13 and second chamber 23. For example, the physical barrier may be a burstable membrane, slidable arm, valve, or the like. For example, in certain embodiments, a burstable membrane initially blocks port 40, which is broken during mixing by pressure created by manipulation of one or more of the plungers back and forth through the barrels. In one embodiment, port 40 comprises a flexible or elastic material with one or more slits, which allow passage of a fluid only upon application of a force.

It should be appreciated that port 40 may be positioned anywhere along the lengths of barrels 10 and 20, such that one or more of the hydrogel components may pass between chambers 13 and 23 via port 40 when mixing. In a particular embodiment, port 40 is positioned at or near the distal end of the first barrel 10 and second barrel 20.

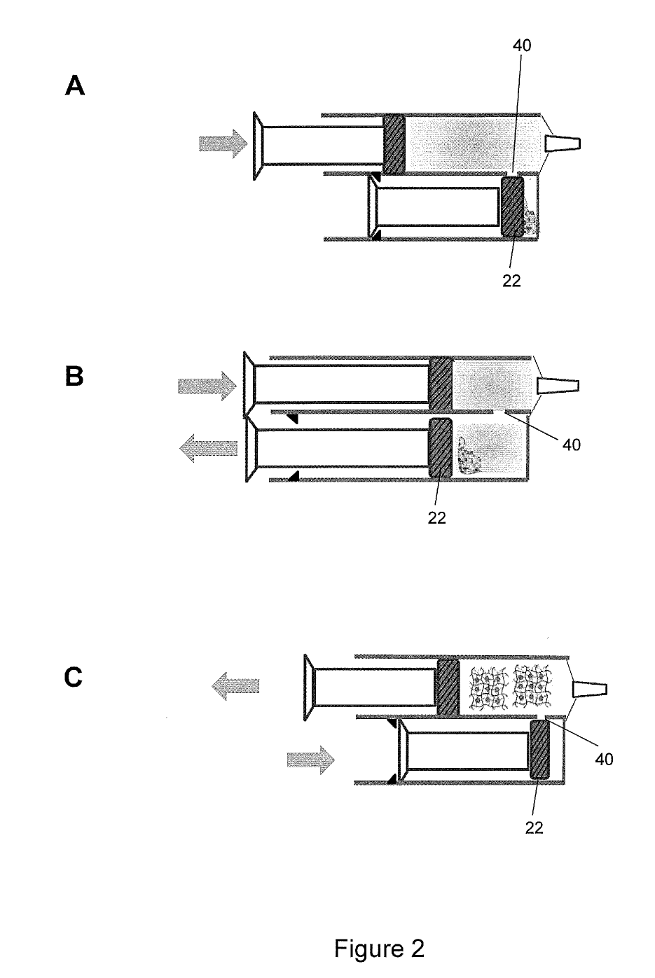

In another embodiment, first stopper 12 and/or second stopper 22 may be guided within first barrel 10 and/or second barrel 20 to block port 40. FIG. 2 depicts an alternate embodiment of device 100, where port 40 is positioned slightly proximal to the distal end of the barrels, such that second stopper 22, in its initial position, physically blocks port 40.

In certain embodiments, port 40 may additionally act as storage chamber for the first component or second component, instead of or in addition to one of the chambers.

In one embodiment, the device comprises a first component of a hydrogel and a second component of a hydrogel, such that when mixed, the two components form a hydrogel. For example, in certain embodiments, the device comprises a solid component and a liquid component. For example, the solid component of the device may be a hydrogel forming compound that is powderized, freeze-dried, desiccated, lyophilized, and the like. In certain embodiments, the solid component comprises a lyophilized solid polymer powder. In one embodiment, the polymer powder is generated by first forming a gel by mixing the polymer, along with any desired therapeutic agents, with a suitable liquid (i.e., distilled water). This mixing process may be aided using a vortex to quicken the mixing process of the solid polymer with the liquid hydrant, with a total mixing time within several minutes. This hydrogel is subsequently freeze-dried over the course of approximately 24 hours to produce a solid polymer. To increase surface area, this polymer is then ground up into smaller fragments to approach a powder. Total preparation time is approximately 24 hours, limited by the lyophilization process.

The liquid component of the device may be any suitable hydrant, including saline or other buffers known in the art. Prior to use of the device to administer the hydrogel, the solid and liquid components are mixed to form a pressure hydrogel. In one embodiment, the solid component, liquid component, or both comprise one or more therapeutic agents.

Hydrogels can generally absorb a great deal of fluid and, at equilibrium, typically are composed of 60-95% liquid and only 5-30% solid polymer. Hydrogels are particularly useful due to the inherent biocompatibility of the cross-linked polymeric network (Hill-West, et al., 1994, Proc. Natl. Acad. Sci. USA 91:5967-5971). Hydrogel biocompatibility can be attributed to hydrophilicity and ability to imbibe large amounts of biological fluids (Brannon-Peppas. Preparation and Characterization of Cross-linked Hydrophilic Networks in Absorbent Polymer Technology, Brannon-Peppas and Harland, Eds. 1990, Elsevier: Amsterdam, pp 45-66; Peppas and Mikos. Preparation Methods and Structure of Hydrogels in Hydrogels in Medicine and Pharmacy, Peppas, Ed. 1986, CRC Press: Boca Raton, Fla., pp 1-27). The hydrogels can be prepared by crosslinking hydrophilic biopolymers or synthetic polymers. Crosslinking of polymers may occur natively, or be induced by chemical or other means. Examples of the hydrogels formed from hydrophilic biopolymers, include but are not limited to, hyaluronans, chitosans, alginates, collagen, dextran, pectin, carrageenan, polylysine, gelatin or agarose. (see.: W. E. Hennink and C. F. van Nostrum, 2002, Adv. Drug Del. Rev. 54, 13-36 and A. S. Hoffman, 2002, Adv. Drug Del. Rev. 43, 3-12). These materials consist of high-molecular weight backbone chains made of linear or branched polysaccharides or polypeptides.

Hydrogels may also be formed from synthetic peptides, which may be specifically designed for particular applications. For example, amphiphilic diblock copolypeptides have been used to form a diblock copolypeptide hydrogel (DCH). In certain embodiments, the solid component of the device comprises an amphiphilic diblock copolypeptide, which when mixed with the liquid component, forms a DCH, sometimes referred to herein as a polypeptide hydrogel. Exemplary amphiphilic diblock polypeptides are known in the art and include, but are not limited to, K180L20, E180L20, K160V40, K180V20, K170L30, and the like.

In one embodiment, the hydrogel of the invention comprises sodium alginate. Sodium alginate is an anionic polysaccharide distributed widely in the cell walls of brown algae, where through binding with water it forms a viscous gum. In extracted form it absorbs water quickly; it is capable of absorbing 200-300 times its own weight in water. Thus, in certain embodiments, the solid component of the device comprises sodium alginate, such that when mixed with the liquid component, forms a sodium alginate hydrogel.

Examples of hydrogels based on synthetic polymers include but are not limited to (meth)acrylate-oligolactide-PEO-oligolactide-(meth)acrylate, poly(ethylene glycol) (PEO), polypropylene glycol) (PPO), PEO-PPO-PEO copolymers (Pluronics), poly(phosphazene), poly(methacrylates), poly(N-vinylpyrrolidone), PL(G)A-PEO-PL(G)A copolymers, poly(ethylene imine), etc. (see A. S Hoffman, 2002 Adv. Drug Del. Rev, 43, 3-12).

In some embodiments, the hydrogel comprises at least one biopolymer. In other embodiments, the hydrogel comprises at least two biopolymers. In yet other embodiments, the hydrogel comprises at least one biopolymer and at least one synthetic polymer.

Hydrogels closely resemble the natural living extracellular matrix (Ratner and Hoffman. Synthetic Hydrogels for Biomedical Applications in Hydrogels for Medical and Related Applications, Andrade, Ed. 1976, American Chemical Society: Washington, D.C., pp 1-36). Hydrogels can also be made degradable in vivo by incorporating PLA, PLGA or PGA polymers. Moreover, hydrogels can be modified with fibronectin, laminin, vitronectin, or, for example, RGD for surface modification, which can promote cell adhesion and proliferation (Heungsoo Shin, 2003, Biomaterials 24:4353-4364; Hwang et al., 2006 Tissue Eng. 12:2695-706). Indeed, altering molecular weights, block structures, degradable linkages, and cross-linking modes can influence strength, elasticity, and degradation properties of the instant hydrogels (Nguyen and West, 2002, Biomaterials 23(22):4307-14; Ifkovits and Burkick, 2007, Tissue Eng. 13(10):2369-85).

Hydrogels can also be modified with functional groups for covalently attaching a variety of proteins, compounds, and therapeutic agents. Hydrogels may also be formed with one or more proteins, compounds, and therapeutic agents embedded within the hydrogel. Molecules or compounds which can be incorporated into or onto the hydrogel include, but are not limited to, vitamins and other nutritional supplements; glycoproteins (e.g., collagen); fibronectin; peptides and proteins; carbohydrates (both simple and/or complex); proteoglycans; antigens; oligonucleotides (sense and/or antisense DNA and/or RNA); antibodies (for example, to infectious agents, tumors, drugs or hormones); and gene therapy reagents. Therapeutic agents which may be linked to, or embedded in, the hydrogel include, but are not limited to, analgesics, anesthetics, antifungals, antibiotics, anti-inflammatories, anthelmintics, antidotes, antiemetics, antihistamines, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, sedatives, sulfonamides, stimulants, sympathomimetics, tranquilizers, urinary anti-infectives, vasoconstrictors, vasodilators, vitamins, xanthine derivatives, and the like. The therapeutic agent can also be other small organic molecules, naturally isolated entities or their analogs, organometallic agents, chelated metals or metal salts, nucleic acids, vectors, peptide-based drugs, or peptidic or non-peptidic receptor targeting or binding agents. In certain aspects, linkage of the therapeutic agent to the polymeric matrix of the hydrogel can be done via a protease sensitive linker or other biodegradable linkage.

In one embodiment, the hydrogel of the invention comprises one or more anesthetics. Exemplary anesthetics include, but are not limited to, proparacaine, cocaine, procaine, tetracaine, hexylcaine, bupivacaine, lidocaine, benoxinate, mepivacaine, prilocalne, mexiletene, vadocaine and etidocaine. The relative amount of the one or more anesthetic in the hydrogel may depend upon, for example, the type of anesthetic, subject being treated, the condition being treated, and location that the hydrogel is administered.

In certain embodiments, one or more multifunctional cross-linking agents may be utilized as reactive moieties that covalently link biopolymers or synthetic polymers. Such bifunctional cross-linking agents may include glutaraldehyde, epoxides (e.g., bis-oxiranes), oxidized dextran, p-azidobenzoyl hydrazide, N-[α.-maleimidoacetoxy]succinimide ester, p-azidophenyl glyoxal monohydrate, bis-[β-(4-azidosalicylamido)ethyl]disulfide, bis[sulfosuccinimidyl]suberate, dithiobis[succinimidyl proprionate, disuccinimidyl suberate, 1-ethyl-3-[3-dimethylaminopropyl]carbodiimide hydrochloride (EDC), N-hydroxysuccinimide (NHS) and other bifunctional cross-linking reagents known to those skilled in the art.

In other embodiments, the hydrogel does not comprise any cross-linking agent. For example, in certain instances, the lack of cross-linking agent may be preferred, as it may offer superior control over delivery of a hydrogel solution to a target site, as described elsewhere herein.

As described herein, the device of the invention isolates the hydrogel forming components prior to the desired mixing and hydrogel formulation. FIG. 1A depicts device 100 prior to use, when port 40 comprises a physical barrier to separate first chamber 13 and second chamber 23, thereby isolating liquid component 14 and solid component 24 from each other. FIG. 1B depicts the preparation of the hydrogel solution by removing the physical barrier of port 40 thereby allowing fluidic communication between first chamber 13 and second chamber 23, and bringing liquid component 14 into contact with solid component 24. In certain embodiments, continual longitudinal manipulation of first plunger 11 and/or second plunger 21 aids in the mixing of liquid and solid components of the hydrogel in order to provide a well-mixed and dispersed hydrogel solution. In certain embodiments, device 100 comprises one or more filters, screens, sieves, or other structures positioned within either barrel 10 and 20, or port 40, to assist in the mixing of the hydrogel solution. Once the hydrogel solution is sufficiently mixed, one of first plunger 11 or second plunger 21 is manipulated such that all of the hydrogel solution is forced into either first chamber 13 or second chamber 23. Once all of the solution is forced into either first chamber 13 or second chamber 23, the physical barrier of pore 40 may be restored.

For example, as depicted in FIG. 1C, substantially all of the hydrogel solution is forced into first chamber 13. Second stopper 22 is guided such that it occludes pore 40, thereby restoring a physical barrier separating first chamber 13 and second chamber 23. In other embodiments, a slidable arm, valve, and the like, is used to restore a physical barrier within the port, thereby isolating first chamber 13 and second chamber 23.

In certain embodiments, device 100 comprises a locking mechanism that prevents movement of at least one of first plunger 11 or second plunger 21 before time-of-preparation, after mixing of components, and/or during application of the solution or gel. Any locking mechanism known in the art may be used. For example, first barrel 10 and/or second barrel 20 may comprise one or more internal teeth, grooves, detents, ridges, rings, and the like, which prevent longitudinal and/or rotational manipulation of the plunger. For example, FIG. 1 depicts device 100 comprising one or more teeth 50 that protrude from the inner wall of second barrel 20, which can be used as a locking mechanism to prevent movement of the second plunger 11. In one embodiment, device 100 comprise one or more locking mechanisms external to either first barrel 10 or second barrel 20. Exemplary external locking mechanisms, include but are not limited to snap-in hook latches, pins, rings, and the like. In certain embodiments, once the hydrogel solution is forced into either first chamber 13 or second chamber 23, one of first plunger 11 or second plunger 21 is locked at a particular location which prevents further manipulation of the plunger or flow of the hydrogel solution out of its resident chamber.

Device 100 can then be used to administer the hydrogel solution by sliding first plunger 11 towards the distal end, thereby forcing the hydrogel solution out of outlet 30, which is positioned at the distal tip of first chamber 13. Outlet 30 may be of any suitable bore which allows for flow of the solution and/or gel out of first chamber 13. It should be understood that while FIG. 1C depicts outlet 30 at the distal tip of first chamber 13, outlet 30 may be positioned at any suitable location along either first chamber 13, second chamber 23, or both. In certain embodiments, outlet 30 is capped to prevent leakage of the liquid component, solid component, and/or hydrogel solution out of outlet 30 during storage or mixing. Outlet 30 is capable of connecting to standard blades or devices such as co-axial needles, mixing valves, and other devices commonly used with syringe-type devices. Outlet 30 may connect to standard devices using universal standards such as a Luer lock or more unconventional attachment methodologies. Outlet 30 may comprise a simple straight outlet or possess other designs, including, for example a sealed or sealable outlet to prevent premature output, shape-based outlet providing flow rate control, etc. In certain embodiments, outlet 30 is configured to mate with a delivery instrument, such as needle or catheter, used to access the target site and administer the mixed solution and/or gel to the target site. For example, the device of the invention can be attached to a core needle and guide needle in order to guide the delivery of the solution and/or gel to a target site. The device may be attached to the needle or catheter prior to or following admixing the solid and liquid components. The needles can be co-axial needles, introducer needles or any other needles with a hollow interior which will allow delivery of the hydrogel solution to the target site. The caliber of exemplary needles may range from about 25 Gauge (G) to 5 G, but are not limited to this range. The hydrogel solution can be injected through catheters or sheath needles including Yueh catheters with a hollow interior to allow delivery of the hydrogel solution. The size of catheters varies and range from 3 French to higher sizes.

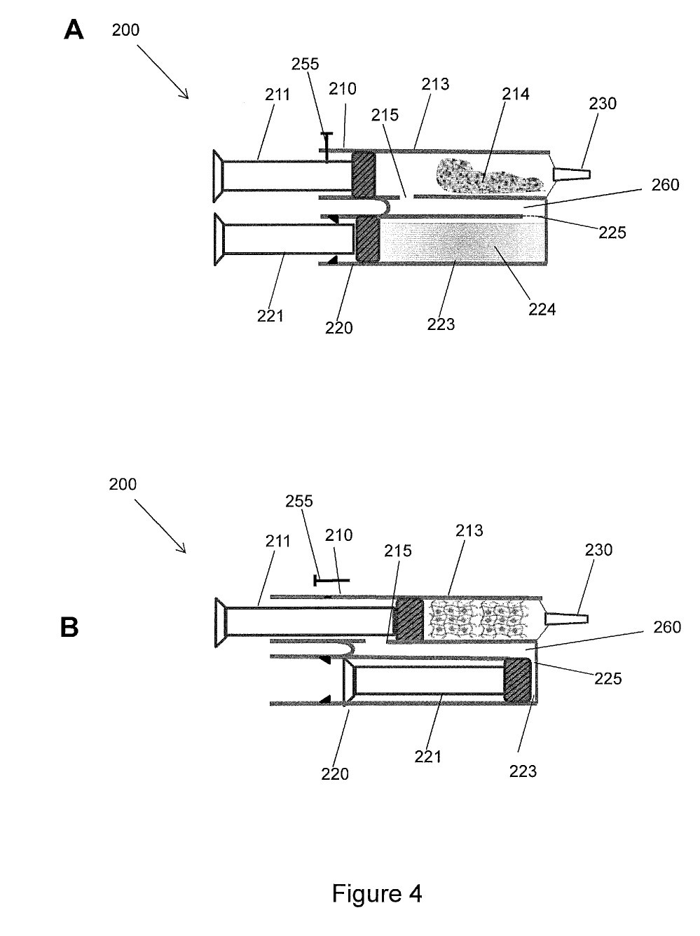

FIG. 4 depicts an alternate embodiment of the device of the invention, where device 200 comprises a third chamber 260 separating first chamber 213 and second chamber 223. Prior to use, first chamber 213 is vacuum sealed and holds a solid component 214 in a vacuum, while second chamber 223 holds a liquid component 224. Both first plunger 211 and second plunger 221 are fully withdrawn towards the proximal end of the first barrel 210 and second barrel 220. First barrel 210 comprises a first hole or port 215 in the barrel wall connecting first chamber 213 to third chamber 260. Similarly, second barrel 220 comprises a second hole or port 225 in the barrel wall, thereby connecting second chamber 223 to third chamber 260. Each hole 215 and 225 may comprise a removable physical barrier, as described above, which restricts passage of a component or solution to and from the chambers. In certain embodiments, device 200 comprises a pin 255, which is used as a locking mechanism of first plunger 211 to prevent movement of first plunger 211 prior to mixing or hydrogel administration. In order to formulate the hydrogel solution, plunger 221 of device 200 is depressed, thereby forcing liquid component 224 through third chamber 260 and into contact with solid component 214 in first chamber 213. Second plunger 221 may be locked into place. In one embodiment, device 200 is shaken to aid in the mixing of the solution. Pin 255 may be manually removed in order to depress first plunger 211 during mixing or administration. Once the solution is formed, it may then be administered through outlet 230.

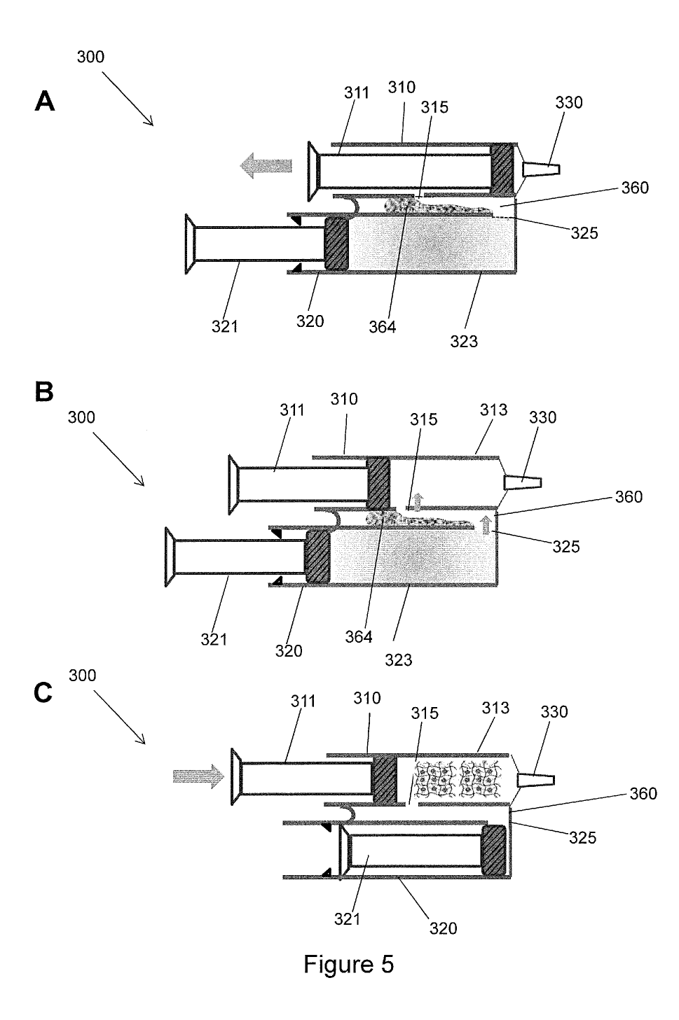

FIG. 5 depicts an alternate embodiment of the device of the invention, where device 300 comprises a third chamber 360 separating first chamber 313 and second chamber 323, but where third chamber 360 initially holds the solid component 364. Prior to use, one or both of first chamber 313 and second chamber 323 holds a liquid component 324. First barrel 310 comprises a first hole or port 315 in the barrel wall connecting first chamber 313 to third chamber 360. Similarly, second barrel 320 comprises a second hole or port 325 in the barrel wall, thereby connecting second chamber 323 to third chamber 360. Each hole 315 and 325 may comprise a removable physical barrier, as described above, which restricts passage of a component or solution to and from the chambers. In one embodiment, first plunger 311 is initially fully depressed distally into first barrel 310. In order to formulate the hydrogel solution, first plunger 311 is moved proximally, thereby enlarging first chamber 313, and second plunger 321 is depressed distally, thereby forcing liquid component 324 through third chamber 360 and into contact with solid component 364. In one embodiment, device 300 is shaken to aid in the mixing of the solution. Once the solution is formed, it may then be administered through outlet 330.

In certain embodiments, the device is pre-packaged and is constructed for single use. For example, the device may be packaged such that the liquid component and solid component are already present at their desired quantities within their respective chambers. In another embodiment, the device may be loaded with desired quantities of liquid and solid components by a user. In certain embodiments, the device is stable at room temperature for extended periods, thereby displaying long shelf-life. Additionally, the hydrogel composition formed in the device of the invention can be customized for one or more parameters including, but not limited to, stiffness and drug release rate, depending particular application of the hydrogel and the patient to which the hydrogel is administered. In one embodiment, the hydrogel of the invention has a stiffness of about 125 Pa to about 225 Pa.

The properties of the hydrogel solution and resultant gel must be carefully designed to ensure efficient delivery of the solution and/or gel to a target site as well as appropriate sealant strength and drug diffusion when applied to the target site. For example, the viscosity of the formed solution must be such that it is able to be injected out of the outlet of the device and through the needle to the target site. Thus, in certain embodiments, too much of a solid component would hamper the deliverability of the solution. However, when applied to the treatment site, the formed hydrogel must exhibit sufficient sealant strength to remain at the target site and completely seal a wound or tissue defect. Thus, in certain embodiments, too little of a solid component would hamper the ability of the hydrogel to effectively treat the defect.

In certain embodiments, the hydrogel formed and administered by the device of the invention is 0.1-20% (wt/vol) solid component. In one embodiment, the hydrogel formed and administered by the device of the invention is 2-10% (wt/vol) solid component. In one embodiment, the hydrogel formed and administered by the device of the invention is 7% (wt/vol) solid component.

In one embodiment, the present invention includes a hydrogel tissue sealant. In certain embodiments, the hydrogel is biocompatible. In one embodiment, the hydrogel compositions are also bioabsorbable. As described elsewhere herein, the hydrogel may comprise any suitable compounds including synthetic compounds and biopolymers. In one embodiment, the hydrogel comprises one or more therapeutic agents that are released to a target site. Exemplary therapeutic agents include, but are not limited to, pain relievers, anesthetics, analgesics, steroids, hormones, anti-inflammatories, anti-coagulants, and the like. For example, in one embodiment, the hydrogel comprises lidocaine. The hydrogel may be used as a tissue sealant for treatment of a tissue defect in any target tissue, including skin, lung, and the like.

The present invention provides a method for treating a tissue defect at a target site within a subject. The method comprises forming a hydrogel solution from a liquid component and solid component, advancing a delivery instrument to the target site, and using the delivery instrument to administer the hydrogel solution to the target site. In certain instances, the method comprises attaching the device of the present invention, as described elsewhere herein, to a delivery instrument, such as a needle or catheter, which is used to administer to the hydrogel percutaneously to a target site. In one embodiment, the hydrogel is delivered directly to a target site during a surgical procedure, where the tissue may be exposed.

The invention provides a method for treating a tissue defect in any tissue of the body, including but not limited to skin, lung, heart, vasculature, liver, kidney, muscle, and the like. Exemplary tissue defects include, but are not limited to cuts, punctures, wounds, burns, ulcers, and the like. In one embodiment, the method is used in conjunction with a biopsy, tissue ablation, or other clinical procedure on tissues such as, lung, kidney, liver, and the like. In certain aspects, the method prevents leakage of a fluid (e.g, air, blood, etc.) from the tissue defect.

In certain embodiments, the method is used to seal a tissue defect in the lung, thereby preventing leakage of air or other gases from the lung. The method may be used to seal a lung defect caused by disease, trauma, medical procedure (e.g., biopsy), or the like.

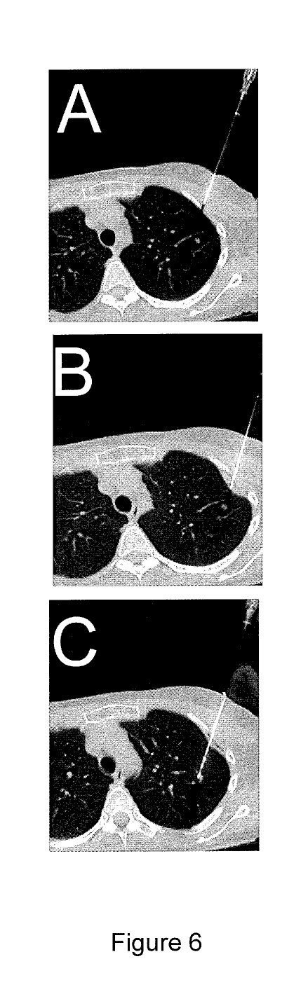

In one aspect, the method comprises preventing a tissue defect by administering a hydrogel solution on a target site prior to the formation of a tissue defect. For example, in one embodiment, the method comprises administering a hydrogel to the surface of a lung, prior to puncture of the lung during a fine needle aspiration biopsy procedure. Lung biopsy is a procedure performed to obtain tissue from the lung. This procedure is commonly performed under CT scan guidance. In one embodiment, the method comprises forming a hydrogel solution in the device of the invention, as described elsewhere herein. The device is attached to a needle, which is guided to the surface of the lung using techniques known in the art (FIG. 6A). The composition is applied to the lung surface at a target site at which a biopsy needle will access the interior of the lung (FIG. 6B). Any suitable amount of the hydrogel solution or gel may be applied to the target site. The particular amount may depend upon the patient, location of the target site, type of procedure being performed, and user preference. In certain embodiments, about 1-20 mL of hydrogel solution is applied to the lung surface. The hydrogel may begin to gel prior to, during, or following application to the lung surface. Once the solution or gel is injected, then the needle is further advanced through the solution and/or hydrogel and into the lung to target the nodule (FIG. 6C). The solution or hydrogel may be administered at any time point prior to the biopsy procedure. The hydrogel will provide a pressure seal once the needle is introduced into the lung. As described elsewhere herein, the hydrogel may be used as a delivery mechanism for 1% lidocaine or bupivacaine to provide pleural anesthesia for an integrated delivery and longer-lasting pain relief. After the tissue sample is taken and assuming no complications (e.g. pneumothorax, severe bleeding) occur, the needle is withdrawn from the lung. Upon retraction of the biopsy needle, the hydrogel seals the puncture formed the biopsy needle, thereby preventing leakage of air or other gases out of the lung, and reducing the risk for pneumothorax. The hydrogel is absorbed into the body after healing of the puncture site has occurred.

In one embodiment, the present invention provides a method for sealing a biopsy or ablation tract in an organ, such as a kidney or liver. Biopsy and ablation of tissue including the liver and kidneys is a growing practice. The administered hydrogel can be used to provide a seal along the biopsy and ablation tract to avoid bleeding, provide prolonged anesthesia, and or/or deliver one or more therapeutic agents. For example, the solution and/or hydrogel may be administered directly into a biopsy tract upon retraction of a needle or catheter from the tissue, thereby plugging the tract. Additionally, the solution and/or hydrogel may be administered such that the composition flows into a biopsy tract.

In one embodiment, the present invention provides a method for separating tissue during dissection. During biopsy and ablation, it is sometimes necessary to obtain a safe plane for the passage of the needle into the target lesion. This passage can be created using the hydrogel administered by way of the present invention. Also, to separate organs like the heart or bowels from targeted tissue, the applied hydrogel of the invention can be used to provide a temporary but stiff barrier for the duration of the procedure.

In one embodiment, the present invention provides a method for delivery of particulate material, therapeutic agents, beads, and the like for intravascular plugging or occlusion.

In one embodiment, the present invention provides a method for delivery of a temporary supportive material. For example, the hydrogel solution may be administered to provide a soft, biodegradable hydrogel substrate to provide temporary support to a treatment site in need thereof. For example, the hydrogel solution may be used in spinal procedures, provide a strong pressurized support that will eventually biodegrade, which also allows for integrating localized therapeutic delivery.

The present invention comprises kits for sealing of body tissue. The kits are manufactured using medically acceptable conditions and contain precursors that have sterility, purity and preparation that is pharmaceutically acceptable. The kit may contain an applicator as appropriate, as well as instructions. A therapeutic agent may be included pre-mixed or available for mixing. Solvents/solutions may be provided in the kit or separately, or the components may be pre-mixed with the solvent. The kit may include syringes and/or needles for mixing and/or delivery.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention. The following working examples therefore, specifically point out the preferred embodiments of the present invention, and are not to be construed as limiting in any way the remainder of the disclosure.

### Example 1

### DCH and Gelatin Hydrogels with Lidocaine

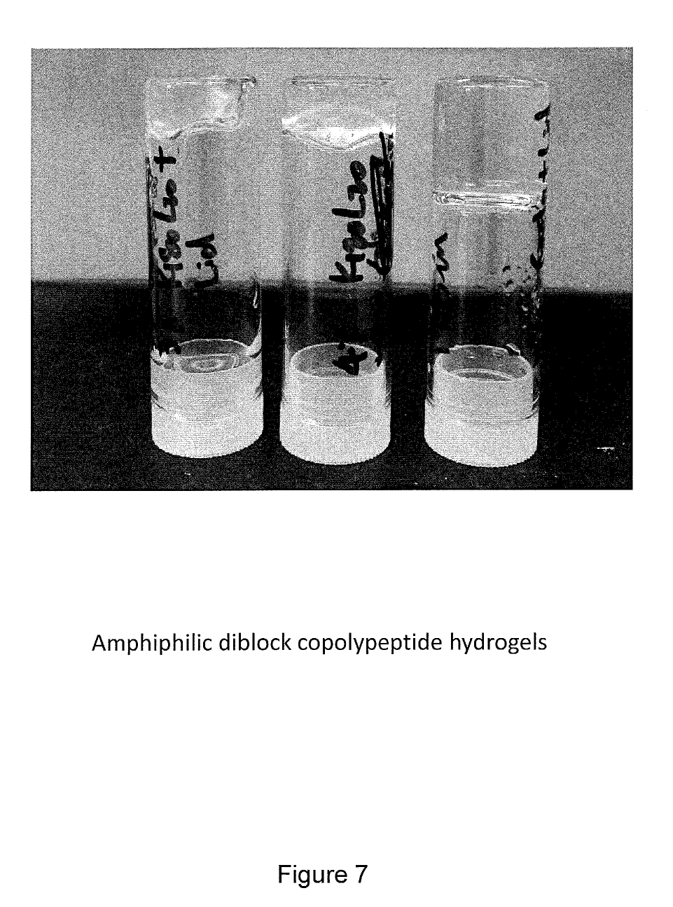

Amphiphilic diblock copolypeptide hydrogels (DCHs) comprising lidocaine were manufactured using different concentrations of K180L20, and were compared to hydrogels made with gelatin. Saline buffer was used to formulate these gels. FIG. 7 depicts three compositions of the hydrogel: Left: 3% K180L20+1% lidocaine (99.7 pa); Middle: 4% K180L20+1% lidocaine (170.7 pa); Right: 10% gelatin+1% lidocaine (233 Pa). In vitro and in vivo experimentation of hydrogel compositions are performed to determine the optimum composition that provides the stiffness level, tissue adherence, pressure, sealing ability, and lidocaine release rates required for lung needle biopsy.

### Example 2

### HA Hydrogels

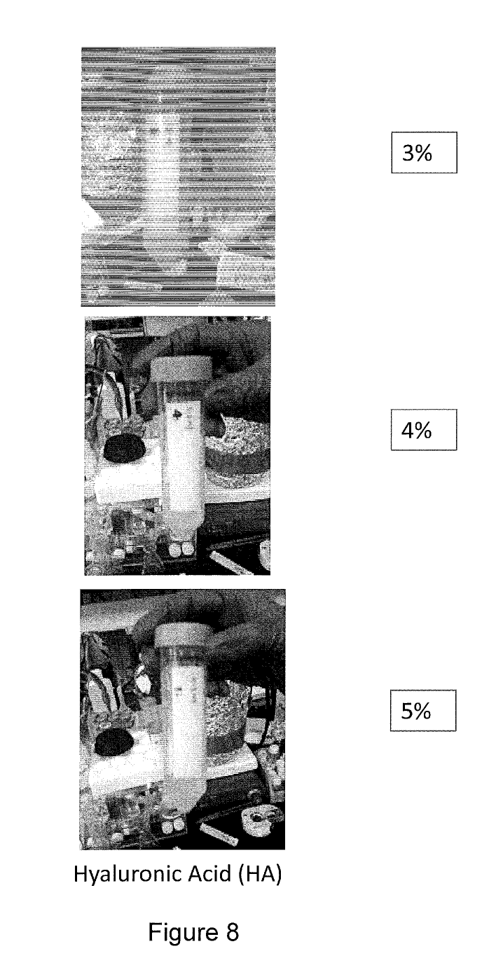

Hyaluronic Acid (HA) based hydrogels have been demonstrated in the past for in vivo use and is FDA approved for various applications. Three concentrations of HA-based hydrogels were prepared for injection, 3% (wt/vol), 4% (wt/vol), and 5%, (wt/vol) by mixing the appropriate amount of HA with saline buffer. The HA Gel reached the stiffness of choice. However the presence of chemical linking within the Gel did not allow for the Gel to be injected through the co-axial needle. The HA Gel tends to remain bonded and clumped in the tube (FIG. 8).

### Example 3

### Hydrogel Stiffness

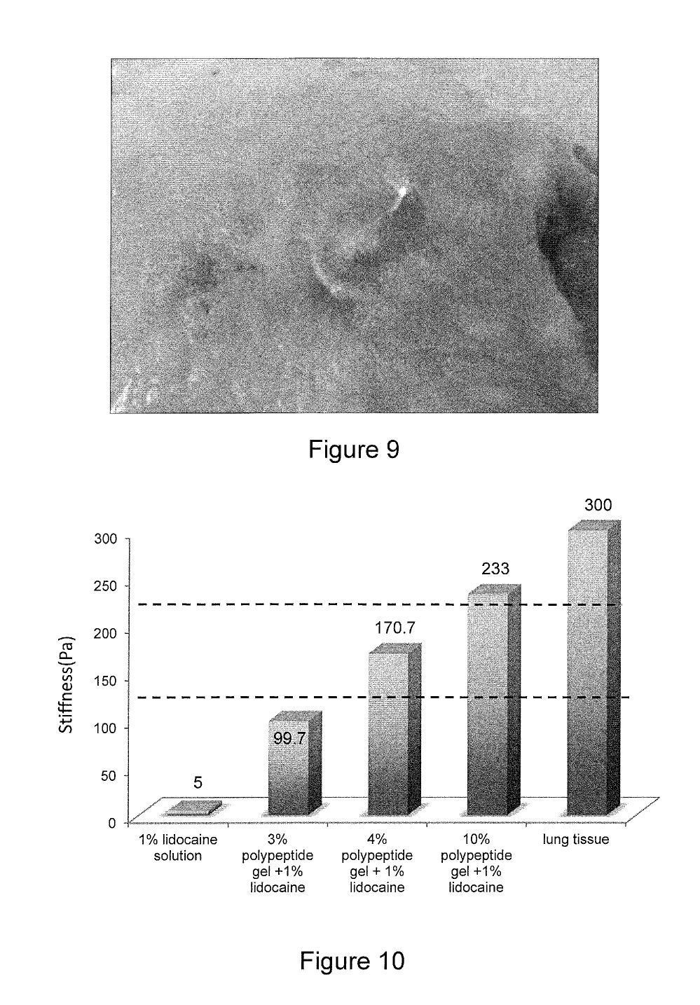

The hydrogel of the invention is required to have an optimum stiffness to allow injection without significant resistance by the operator/interventionist, and the ability to stay in location without significant dispersion away from the target zone. To test the appropriate stiffness, various concentration of DCH at 1% (wt/vol), 3% (wt/vol), and 4% (wt/vol) and gelatin at 6% (wt/vol) and 10% (wt/vol) were injected through a coaxial needle to subjectively determine the approximate range of usable gel stiffness. The concentrations of 1% to 4% were injected without significant resistance. The 6% and 10% concentrations were felt to be of higher resistance and difficult to use for clinical practice. The DCH was then injected in vitro into the fascia plane between chicken breast muscle and skin. The 1% DCH demonstrated quick dispersion, while DCH at 3% and above remained in place adequately (FIG. 9). Through these experiments it was deducted that 3% and 4% DCH are of appropriate stiffness; allow injection through typical coaxial needles; and possessed limited dispersion in tissue. The stiffness of 3% and 4% DCH was measured using a rheometer, and corresponds to the 125 to 225 Pascal (Pa) pressure range (FIG. 10).

### Example 4

### Lidocaine Dispersion

The hydrogel of the invention can be used as a medium for drug delivery. The advantages of using the hydrogel comprising one or more anesthetics such as e.g., lidocaine, are to allow for prolonged anesthesia throughout the procedure and to avoid multiple injections during the procedure. Here the hydrogel is formulated with 1% (wt/vol) lidocaine, an FDA approved and commonly used drug to provide anesthesia during procedures.

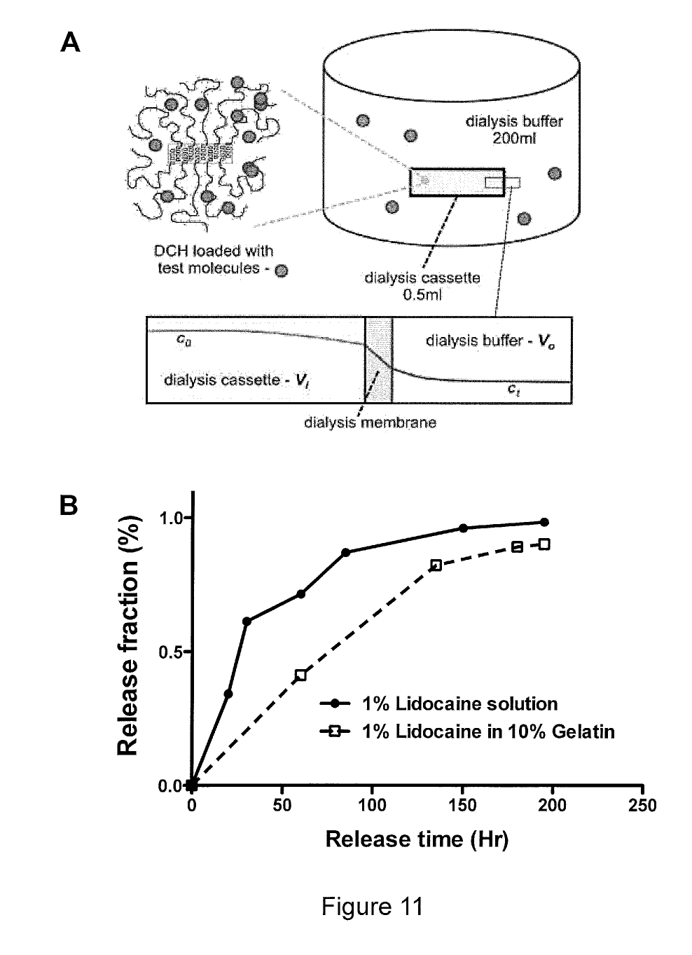

The release profile of lidocaine from formulated hydrogels was investigated. The hydrogel demonstrated the capability for gradual prolonged release of lidocaine over an extended period of time (FIG. 11). The release duration and release rate of an embedded therapeutic agent, such as lidocaine, may be controlled or optimized by tuning the concentration and/or composition of hydrogel.

### Example 5

### Mixing Mechanism Validation

To quantify the mixing time using a mechanical push-pull mechanism as described above, experiments were conducted using two syringes connected using a three-way valve. In initial experiments conducted with the alginate powder in one syringe and normal saline in the other syringe, mechanical mixing via pushing each syringe head in turn required at least 15-20 rounds of pushing to form a homogeneous gel. It is noted that the powder adhered to the surface of the syringe and the mixing motion effectively caused minimal “sliding” of the liquid across the surface of a layered powder. Additionally, the formation of a large number of bubbles during the mixing process was observed.

In an attempt to reduce the amount of mechanical mixing and bubbles by maximizing the surface area of the solid, a pre-formed diluted hydrogel (3.5% (wt/vol)), with and without 1% (wt/vol) lidocaine included, was freeze-dried to demonstrate the encapsulation effect, and mixed the resultant crystalline solid with normal saline. This experiment significantly increased the amount of mixing required, with continuous pushing for approximately 10 minutes necessary to produce a homogeneous gel.

To create smaller fragments that will easily spread throughout the liquid hydrant during the initial mechanical mixing movements, the freeze-dried hydrogel was ground to reduce the bulky polymer into smaller pieces, thereby breaking the crystalline structure. Mixing experiments using the ground-up freeze-dried powder resulted in a homogeneous 7%(wt/vol) gel after only 5 rounds of pushing the plungers back and forth. This suggests that, after preparation, this gel is capable of providing a complete mixing process within several seconds using a push-pull mechanical mixing procedure.

### Example 6

### Alginic Acid (Sodium Alginate) Hydrogel

Sodium alginate is an anionic polysaccharide distributed widely in the cell walls of brown algae, where through binding with water it forms a viscous gum. In extracted form it absorbs water quickly; it is capable of absorbing 200-300 times its own weight in water. Various sodium alginate hydrogels, each comprised of a different concentration of sodium alginate, were formulated and compared, as described below.

To determine the stiffness of sodium alginate hydrogels, for injection through small gauge biopsy needles (e.g., 19 G) without significant resistance, four preparations of sodium alginate hydrogels at 4% (wt/vol), 6% (wt/vol), 7% (wt/vol), and 8% (wt/vol) were tested by an interventional radiologist with extensive experience in thoracic procedures, blinded to the concentrations. The radiologist injected the four concentrations of hydrogels through a 19G and 10.2 cm length co-axial needle system using a syringe. The 4%, 6%, and 7% hydrogels were easily injected through the coaxial needle with increasing resistance, but acceptable based on the operators' experience in clinical practice and setting. The 8% hydrogel was found to be too stiff and resistive to injection. The gels injected were also dropped on a flat surface and observed for dispersion for 10 minutes. 8% had the least dispersion. 7% remained in position for period of observation. The dispersion of the 4% and 6% hydrogel was rapid and was deemed inadequate to hold in place to perform the intended function. Therefore, the 7% sodium alginate hydrogel is deemed most useful for injection with limited resistance and dispersion.

Further experiments determined the stiffness of the 7% sodium alginate hydrogel, with 1% lidocaine, to be 160 Pa, while the 7% sodium alginate hydrogel, without lidocaine, was 165 Pa for 7% gel. Comparatively, 1% lidocaine in saline corresponds to approximately 0.1 Pa.

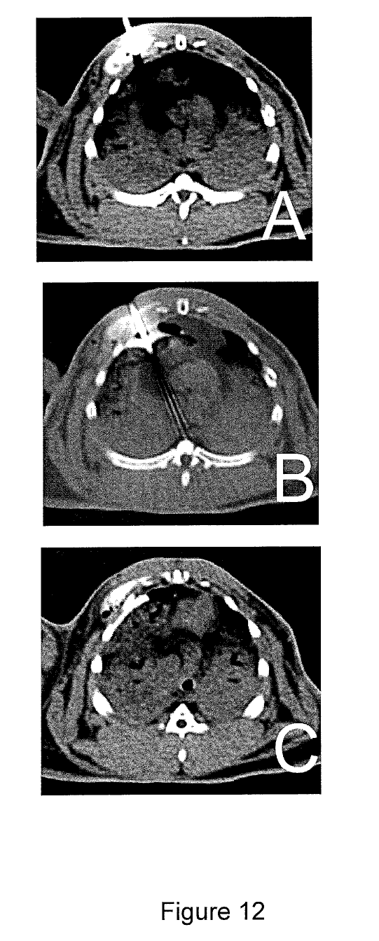

The ability to inject the 7% sodium alginate hydrogel in tissue was tested on a rabbit carcass. The animal was placed in a CT scanner and, under CT guidance, a 19G coaxial needle was placed at the extra pleural surface. Then up to 5 cubic centimeters (cc) of the hydrogel was injected (FIG. 12A and FIG. 12B). Thereafter, the animal was scanned for 10 minutes.

No significant resistance when injecting the 7% sodium alginate hydrogel was observed in excess of clinically gained experience with human subjects. The hydrogel dispersed to the surface of the pleura but did not leak into the lung or disperse in the pleural surface. This suggests that the hydrogel was able to hold up well without dissemination (FIG. 15B). On follow-up CT scan, the hydrogel maintained position for 10 minutes following injection (FIG. 12C).

Experiments are conducted to evaluate the dispersion of the hydrogel in target tissue over time; to evaluate safety of the hydrogel in mammalian tissue, in particular for the pleura and lung; and, to evaluate the efficacy of the hydrogel in controlling/preventing pneumothorax. A rabbit model is used for these experiments.

To evaluate the safety of the hydrogel in tissue and to measure dispersion, each rabbit is injected with a 7% sodium alginate hydrogel on one side of the hemithorax and subcutaneously at the level of the shoulder blades. The hydrogel is injected on the ride side of the animals. The animals are sacrificed in 48 hours to evaluate for local inflammatory response and adverse tissue effect.

To evaluate the efficacy of the hydrogel, two sets of animals are identified, each set containing 10 rabbits. The right lung is punctured with a 19G needle. One set of subjects undergo injection at the pleura before insertion of the needle through the pleura into the lung. The other set of subjects undergo insertion through pleura into the lung without pre-application of the hydrogel. Thereafter the animals are followed with serial chest radiographs at 1 hour, 2 hours, 4 hours, 12 hours, 24 hours and 48 hours following the needle insertion. At 48 hours, a CT scan is performed and then the animals are sacrificed for aforementioned safety and dispersion analyses.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A device for sealing a tissue defect in a body tissue of a subject, wherein the device comprises:
a first barrel and a first plunger engaged with the proximal end of the first barrel, thereby forming a first chamber within the first barrel, wherein the first chamber houses a first component of a hydrogel;
a second barrel and a second plunger engaged with the proximal end of the second barrel, thereby forming a second chamber within the second barrel chamber, and wherein the second chamber houses a second component of a hydrogel; and
at least one port connecting the first chamber and second chamber;
wherein the contents of the first chamber and second chamber are temporarily isolated to prevent contact of the first component and second component prior to mixing.

2. The device of claim 1, wherein the port is configured for control by a user to allow communication between the first chamber and second chamber, thereby allowing contact of the first component and second component to form a hydrogel solution.

3. The device of claim 1, further comprising at least one outlet positioned on at least one of the first chamber and second chamber.

4. The device of claim 1, wherein the outlet is configured to mate with a delivery instrument, selected from the group consisting of a needle and a catheter.

5. The device of claim 1, wherein at least one of the first component and second component comprises one or more therapeutic agents.

6. The device of claim 1, wherein the one or more therapeutic agents comprise one or more anesthetics.

7. The device of claim 1, wherein at least one of the first component and second component comprises a powderized and lyophilized hydrogel.

8. The device of claim 1, wherein at least one of the first component and second component, comprises at least one of the group consisting of sodium alginate, hyaluronic acid, gelatin, fibrin, collagen, laminin, synthetic amphiphilic diblock copolypeptide, and agarose.

9. The device of claim 2, wherein the hydrogel solution forms a hydrogel capable of being injected to a target site of the subject.

10. The device of claim 2, wherein the hydrogel solution forms a hydrogel, which remains at a target site of the subject, thereby sealing a tissue defect present at the target site.

11. A method of sealing a tissue defect at a target site of body tissue of a subject, the method comprising
providing a device comprising:
a first barrel and a first plunger engaged with the proximal end of the first barrel, thereby forming a first chamber within the first barrel, wherein the first chamber houses a first component of a hydrogel;
a second barrel and a second plunger engaged with the proximal end of the second barrel, thereby forming a second chamber within the second barrel chamber, and wherein the second chamber houses a second component of a hydrogel; and
at least one port connecting the first chamber and second chamber;
wherein the contents of the first chamber and second chamber are temporarily isolated to prevent contact of the first component and second component prior to mixing;
mixing the first component and second component to form a hydrogel;
attaching the device to a delivery instrument;
guiding the delivery instrument to target site; and
administering the hydrogel from the device, through the delivery instrument, onto the target site.

12. The method of claim 11, wherein the body tissue is selected from the group consisting of lung, liver, kidney, bone, and vasculature.

13. The method of claim 11, wherein the tissue defect is a puncture wound formed during a clinical procedure.

14. The method of claim 11, wherein administering the hydrogel onto the target site is done prior to the development of a tissue defect at the target site.

15. The method of claim 11, wherein at least one of the first component and second component comprises one or more therapeutic agents.

16. The method of claim 11, wherein the one or more therapeutic agents comprise one or more anesthetics.

17. The method of claim 11, wherein at least one of the first component and second component comprises a powderized and lyophilized hydrogel.

18. The method of claim 11, wherein at least one of the first component and second component, comprises at least one of the group consisting of sodium alginate, hyaluronic acid, gelatin, fibrin, collagen, laminin, synthetic amphiphilic diblock copolypeptide, and agarose.

19. The method of claim 11, wherein the administered hydrogel remains at a target site of the subject, thereby sealing a tissue defect present at the target site.

20. A composition for sealing a tissue defect in a body tissue of a subject, the composition comprising a hydrogel comprising 7% (wt/vol) sodium alginate and 1% (wt/vol) lidocaine.
